(19) United States
(12) Patent Application Publication
(10) Pub. No.: US 2005/0137556 A1
(43) Pub. Date: Jun. 23, 2005

(54) Sanitary absorbent article having a reinforced structure

(76) Inventor: Henri Brisebois, Lachenaie (CA)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/994,951

(22) Filed: Nov. 22, 2004

Related U.S. Application Data
(60) Provisional application No. 60/526,793, filed on Dec. 4, 2003.

(51) Int. Cl. A61F 13/15; A61F 13/20

(52) U.S. Cl. 604/385.04

(57) ABSTRACT

The present invention provides a sanitary napkin comprising a main body, at least one flap and a flap reinforcing element. The main body includes a fluid-permeable layer and a liquid-impervious layer and a pair of opposite longitudinal side edges and opposite transverse side edges. The flap is attached to the main body at an area of juncture, and has two sides and a distal end. The flap and the main body together define a continuous contour that includes a peripheral seal for joining the fluid-permeable layer and the liquid-impervious layer together. The continuous contour comprises a first segment and a second segment. The flap reinforcing element spans across the area of juncture thereby bridging the main body and the flap, and has an edge portion that is co-extensive with the first segment and the second segment. The flap reinforcing element has a boundary that is internal to the peripheral seal.

### CROSS REFERENCE TO RELATED APPLICATIONS

This application claims priority to Application No. 60/526,793 filed on Dec. 4, 2003.

### FIELD OF THE INVENTION

The present invention relates to sanitary absorbent articles, such as disposable sanitary napkins, which feature a reinforced structure.

### BACKGROUND OF THE INVENTION

In general, sanitary absorbent articles are large-scale commercially manufactured articles used to absorb and retain bodily exudates. Such articles, which are convenient since they are economical yet disposable, include sanitary napkins, infant diapers, adult incontinence pads, and the like.

The technology surrounding sanitary absorbent articles, and particularly feminine sanitary napkins, has undergone several advances over the past few decades. One such advance was the addition of a flap projecting laterally from each longitudinal side edge of the article when the article is in a flattened state. Such flaps may be comprised of integral extensions of a material from which the article is formed or, alternatively, they may simply be comprised of additional material added to the article after its formation.

During use, the flaps are folded over the edges of the wearer's undergarment. Thus, they more firmly secure the article to the undergarment, stabilize the article within the undergarment, provide an increased absorptive area for bodily exudates, and help prevent the undergarment from becoming soiled in part by protecting the side edges of the wearer's undergarment. The flap concept has been well received in the marketplace and, as a result, articles featuring flaps of various configurations and conformations are currently available to the consumer.

Conventional wisdom on the part of both designers and consumers has to date dictated that the flaps on absorbent articles should be maximized at their area of juncture with the main body of the article in order to provide a greater area of protection against exudate leakage over the side of the article. Hence, a very common flap configuration is an isosceles (i.e. bilaterally symmetrical) trapezoidal-shaped flap having its base adjoined with the longitudinal side edge of the main body of the article and projecting in tapering fashion away therefrom to the top of the trapezoid. The size and shape of the flaps, however, may vary from article to article.

Sanitary napkins featuring flaps characterized by a width (dimension measured along the longitudinal axis of the sanitary napkin) that increases in a direction away from the main body of the napkin have also been proposed. This flap design offers a number of advantages, an important one being to securely retain the sanitary napkin to the undergarment of the wearer. Such enhanced retention is desirable because it stabilizes the sanitary napkin against the body of the wearer and thus reduces the likelihood of failure events.

One of the difficulties associated with the use of sanitary napkins employing flaps, however, is that the wearer, once the sanitary napkin has been used, will not detach the flaps from the undergarment before lifting away the sanitary napkin's main body. The tendency is to simply pull the main body while the flaps remain attached to the undergarment. This manipulation creates stress levels on the sanitary napkin at the area of juncture main body/flap and may cause the flaps to tear and separate from the main body. This is undesirable since the wearer must then remove the separated flap(s) in a subsequent operation that may cause annoyance. Moreover, the tearing of the flaps may expose the user's undergarment to bodily exudates contained in the sanitary napkin thereby soiling the same.

Considering this background, it clearly appears that there is a need in the industry to develop a feminine sanitary napkin which can better resist flap tearing.

### SUMMARY OF THE INVENTION

Under a first broad aspect, the present invention provides a sanitary napkin adapted to be worn in the crotch portion of an undergarment. The sanitary napkin comprises a main body, at least one flap and a flap reinforcing element. The main body includes a fluid-permeable layer facing toward the wearer when the sanitary napkin is placed in the undergarment, a liquid-impervious layer, an absorbent system for absorbing liquid, a pair of opposite longitudinal side edges and a pair of opposite transverse side edges. The absorbent system is located between the fluid-permeable layer and the liquid-impervious layer. The flap is attached to the main body at an area of juncture, and has two sides and a distal end that is remote from the area of juncture. The flap and the main body together define a continuous contour, wherein at least a portion of the continuous contour includes a peripheral seal for joining the fluid-permeable layer and the liquid-impervious layer together. The continuous contour comprises a first segment and a second segment. The first segment extends along one of the longitudinal side edges from the area of juncture toward one of the transverse side edges and the second segment extends along one of the sides of the flap from the area of juncture toward the distal end. The flap reinforcing element spans across the area of juncture thereby bridging the main body and the flap. The flap reinforcing element has an edge portion that is co-extensive with the first segment and the second segment of the continuous contour, and a boundary that is internal to the peripheral seal.

### BRIEF DESCRIPTION OF THE DRAWINGS

A detailed description of preferred embodiments of the present invention is provided herein below with reference to the following drawings, in which:

FIG. 1 is a top plan view of a first non-limiting example of implementation of a sanitary napkin of the present invention;

FIG. 2a is a cross-sectional view taken along line 2a-2a of FIG. 1;

FIG. 2b is a cross-sectional view of a sanitary napkin constructed in accordance with a first variant of the invention;

FIG. 2c is a cross-sectional view of a sanitary napkin constructed in accordance with a second variant of the invention;

FIG. 2d is a cross-sectional view of a sanitary napkin constructed in accordance with a third variant of the invention;

FIG. 3 is a top plan view of a sanitary napkin constructed in accordance with a fourth variant of the invention;

FIG. 4 is a cross-sectional view taken along line 4-4 of FIG. 3;

FIG. 5 is a top plan view of a sanitary napkin constructed in accordance with a fifth variant of the invention;

FIG. 6 is a cross-sectional view of a sanitary napkin constructed in accordance with a sixth variant of the invention; and

FIG. 7 is a cross-sectional view of a sanitary napkin constructed in accordance with a seventh variant of the invention.

In the drawings, preferred embodiments of the invention are illustrated by way of examples. It is to be expressly understood that the description and the drawings are only for the purpose of illustration and as an aid to understanding. They are not intended to be a definition of the limits of the invention.

### DETAILED DESCRIPTION

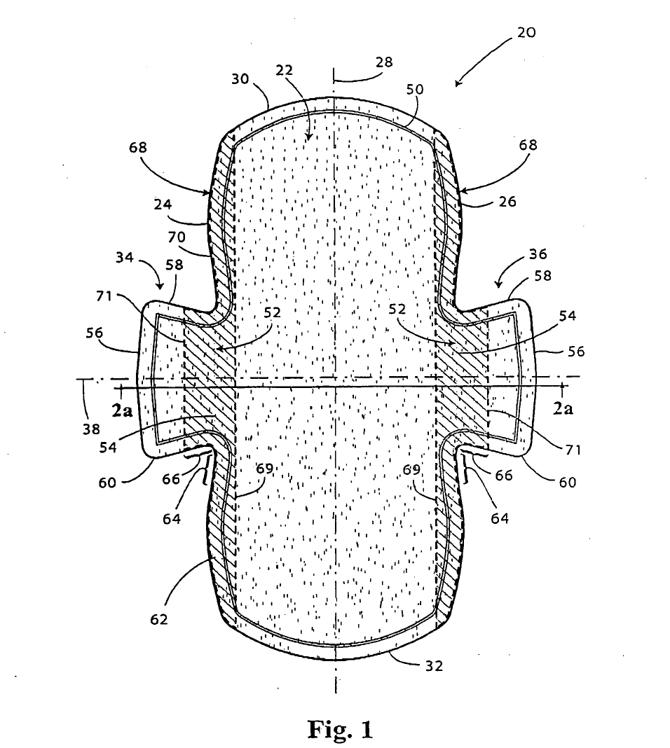

With reference to FIG. 1, there is shown a non-limiting example of implementation of a sanitary absorbent article, a disposable sanitary napkin 20. The sanitary napkin 20 comprises a main body 22 having generally opposing longitudinal side edges 24, 26, and an imaginary longitudinal centerline 28 running down the center of the sanitary napkin 20, generally equidistant from the longitudinal side edges 24, 26.

The main body 22 also has two generally opposing transverse side edges 30, 32. Projecting laterally from each of the longitudinal side edges 24, 26 of the main body 22 are flaps 34, 36. The flaps 34, 36 are generally of the shape of an isosceles (i.e. bilaterally symmetrical) trapezoid, with the shorter of the two parallel sides thereof adjoining the longitudinal side edges 24, 26 of the main body 22 of the napkin 20. An imaginary transverse centerline 38 runs across the sanitary napkin 20 and intersects imaginary longitudinal centerline 28.

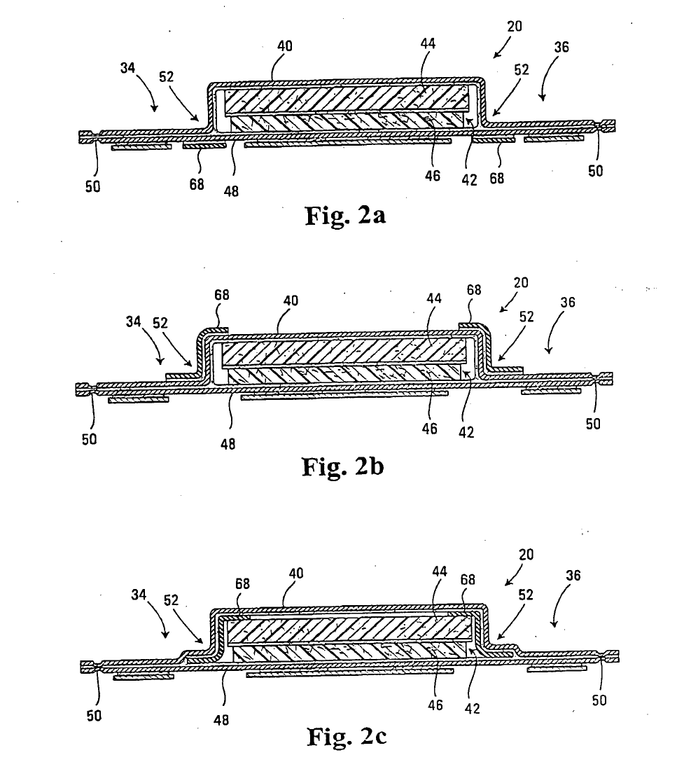

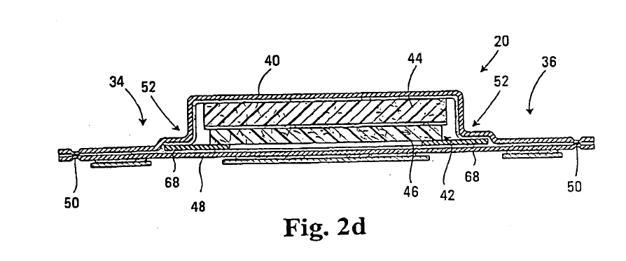

As shown in FIG. 2a, the napkin 20 is a laminate structure. The main body 22 includes a fluid-permeable cover layer 40, which will face the body of a wearer when the sanitary napkin 20 is in use, a liquid-impervious barrier layer 48, which will face the environment (i.e. away from the body of the wearer, and in almost all cases the wearer's undergarment) when the sanitary napkin 20 is in use and an absorbent system 42 located between the fluid-permeable cover layer 40 and the liquid-impervious barrier layer 48. The absorbent system 42 may have a single layer or multiple layers. In the example of implementation depicted in the drawings, absorbent system 42 includes a multi-layer structure. More specifically, the absorbent system 42 has a first layer, called a “transfer layer” 44 immediately underneath the fluid-permeable cover layer 40 and a second layer called an “absorbent layer” 46 between the transfer layer 44 and the liquid-impervious barrier layer 48.

The fluid-permeable cover layer 40 and the liquid-impervious barrier layer 48 are sealed together along their peripheral edges (including the flaps 34, 36) to form a peripheral seal 50, containing the absorbent system 42. The peripheral seal 50 can be realized by using any means commonly known in the art such as by gluing, crimping, pressure and/or heat-sealing, ultrasonics, and the like. Although peripheral seal 50 is shown as being continuous, the present invention also contemplates embodiments where the peripheral seal 50 is intermittent and therefore does not necessarily completely enclose the absorbent system 42. Thus, a variety of seal configurations are within the scope of this invention.

Each of the above layers will now be described in greater detail.

Fluid-Permeable Cover Layer

With reference to FIGS. 1 and 2a, the fluid-permeable cover layer 40 is the top layer of the sanitary napkin 20. The purpose of the fluid-permeable cover layer 40 is to provide an interface that would normally contact the body of the wearer when the sanitary napkin 20 is in use. The fluid-permeable cover layer 40 is porous to liquids since its main function is to capture as quickly as possible a discharge of bodily exudate and transfer it to the absorbent system 42 underneath.

Under one specific example of implementation, the fluid-permeable cover layer 40 is formed from an apertured thermoplastic film. Such films are common in the art. Because of the high porosity of such films, they accomplish the function of quickly transferring body exudate to the inner layers (i.e. the absorbent system 42) of the napkin 20.

The fluid-permeable cover layer 40 can also be made of fibrous materials, such as non-woven fibrous materials. The fluid-permeable cover layer 40 may be composed of only one type of fiber, such as polyester, or may be composed of bicomponent or conjugate fibers having a low melting point component and a high melting point component. Bicomponent fibers may be made up of a polyester core and a polyethylene sheath. The use of appropriate bicomponent materials results in a fusible non-woven fabric. Using a fusible fabric increases the ease with which the fluid-permeable cover layer 40 may be mounted to the barrier layer 48 at the peripheral seal 50.

The fibers may be selected from a variety of natural and synthetic materials such as nylon, polyester, rayon (in combination with other fibers), cotton acrylic fiber and the like, and combinations thereof. It will be evident to the person skilled in the art that a wide variety of other types of non-woven fabric materials can also be used.

Transfer Layer

Adjacent to the fluid-permeable cover layer 40 on its inner side and bonded thereto is the optional fluid transfer layer 44, that may form part of the absorbent system 42. The transfer layer 44 provides the means of receiving body fluid from the fluid-permeable cover layer 40 and holding it until the highly-dense absorbent layer 46 has an opportunity to absorb it.

The transfer layer 44 is, preferably, more dense than than the fluid-permeable cover layer 40 and has a larger proportion of smaller pores than does the latter. These attributes allow the transfer layer 44 to contain body fluid and hold it away from the outer side of the fluid-permeable cover layer 40, thereby preventing the fluid from re-wetting the fluid-permeable cover layer 40 and its surface. However, the transfer layer 44 is preferably not so dense as to prevent the passage of the fluid through the transfer layer 44 and into the underlying absorbent layer 46.

The transfer layer 44 may be composed of fibrous materials such as wood pulp, polyester, rayon, flexible foam, or the like, or combinations thereof. The transfer layer 44 may also comprise thermoplastic fibers for the purpose of stabilizing the layer and maintaining its structural integrity. The transfer layer 44 may be treated with surfactant on one or both of its sides in order to increase its wettability. However, the transfer layer 44 is generally relatively hydrophilic and may not require additional treatment. The transfer layer 44 is preferably bonded on both sides to the adjacent layers, i.e. the fluid-permeable cover layer 40 and the absorbent layer 46.

Absorbent Layer

Immediately adjacent to and bonded to the transfer layer 44 is the absorbent layer 46; the latter also forming part of the absorbent system 42. The absorbent system 42 may comprise a single layer structure or may comprise a multi-layer structure.

The absorbent layer 46 is a highly dense layer having a fine porosity. It has a large liquid-holding capacity and it is extremely retentive. Preferably, the absorbent layer 46 comprises a pulp fluff material and may optionally include other absorbent materials or non-absorbent materials such as conjugate fibers, fusible fibers, binders, sphagnum moss, superabsorbents, and the like and combinations thereof.

Liquid-Impervious Barrier Layer

Underlying the absorbent system 42 is a liquid-impervious barrier layer 48 which comprises liquid-impervious film material such as to prevent liquid that is entrapped in the absorbent layer 46 from egressing the sanitary napkin 20 and staining the wearer's undergarment. The liquid-impervious barrier layer 48 can be made of polymeric film, such as polyethylene or a polyethylene/ethylvinyl acetate (EVA), which are both inexpensive and readily available. The polymeric film is capable of fully blocking the passage of liquid or gas that may emanate from the absorbent system 42. In a variant, breathable films may be used that allow passage of gases while blocking liquid.

Flaps

Preferably, the flaps 34, 36 are comprised of integral continuous extensions of the fluid-permeable cover layer 40 and the liquid-impervious barrier layer 48. Thus, the flaps 34, 36 are dual layer structures, the upper layer being a continuous extension of the fluid-permeable cover layer 40 while the bottom layer is a continuous extension of the liquid-impervious barrier layer 48. It should be specifically understood, however, that flaps having a single layer structure also remain within the scope of the present invention. FIG. 2a further shows that flaps 34, 36 do not contain the absorbent system 42 therein. However, in an alternative embodiment not shown in the drawings, the absorbent system may extend into the flaps or the flaps may be provided with separate absorbent layers.

Since both flaps 34 and 36 are identical, only flap 34 will be described herein.

As depicted in FIGS. 1 and 2a, flap 34 extends from the main body 22 starting at an area of juncture 52. Flap 34 is preferably an extension of the fluid pervious layer and the liquid impervious layer, as mentioned above, but in an alternative embodiment, flap 34 can also be a separate structure that is attached to the main body 22 at the area of juncture 52. The flap 34 has a proximal end 54 that is adjacent the area of juncture 52, a distal end 56 that is remote from the area of juncture 52, as well as a pair of sides 58, 60 which extend between the proximal and distal ends. Preferably, the flaps 34, 36 do not longitudinally extend the entire length of the sanitary napkin 20.

The flaps 34, 36 and main body 22 together delineate a continuous contour 62 which includes at least two contiguous segments, namely a first segment 64 that is defined by a portion of longitudinal side edge 24, and a second segment 66 that is defined by a portion of side 60. First segment 64, more specifically, extends from the area of juncture 52 toward transverse side edge 32 while second segment 66 extends from the area of juncture 52 toward the distal end 56 of the flap. It should be noted that contiguous segments such as those described above exist at each side of each flaps 34, 36.

The sanitary napkin 20 further includes flap reinforcing elements. Various embodiments of the flap reinforcing elements will be described herein below with respect to FIGS. 1-7.

FIGS. 1 and 2a show that sanitary napkin 20 comprises a pair of flap reinforcing elements 68. FIG. 1 is a top plan view in which various layers of the napkin are transparently depicted in order to show the flap reinforcing elements 68 that, in the embodiment depicted in FIG. 1, are attached below barrier layer 48. In FIG. 1, the reinforcing elements 68 are shown in cross-hatching. As shown, both flap reinforcing elements 68 extend in a direction that is substantially parallel to imaginary longitudinal centerline 28. In addition, each flap reinforcing element 68 spans across the area of juncture 52, thereby bridging the main body 22 and a respective flap. By “bridging”, it is meant that each flap reinforcing element 68 respectively extends from a flap 34 or 36 into main body 22. Each flap reinforcing element 68, by bridging the main body 22 and its respective flap 34 or 36, reinforce the sanitary napkin 20 along the area of juncture 52 such that it is better able to resist the stress levels that occur when a user grasps a used napkin and attempts to remove it from an undergarment without having previously detached the flaps.

As is also shown in FIG. 1, each flap reinforcing element 68 is generally shaped such that a portion of an outer edge 70 of the flap reinforcing element 68 is co-extensive with the continuous contour 62 and, more particularly, with the first and second segments 64, 66. By “co-extensive”, it is meant that the portion of the outer edge 70 of each flap reinforcing element 68 shares a common boundary with the continuous contour 62 defined by flaps 34, 36 and main body 22.

Although FIGS. 1 and 2a show that each flap reinforcing element 68 fully extends in a longitudinal direction between both transverse side edges 30 and 32, it should be specifically understood that embodiments in which a flap reinforcing element does not fully extend between both transverse side edges 30, 32 remain within the scope of the present invention.

In the specific embodiment shown in FIG. 1, each flap reinforcing element 68 includes two boundaries 71 and 69 that are shown in dotted lines in FIG. 1. The boundary 71 is located in an area which is intermediate the distal ends 56 of flaps 34, 36 and their respective proximal ends 54, and the boundary 69 is located between the two areas of juncture 52 of the two flaps 34 and 36. Both boundaries 71 and 69 have portions that are internal to the peripheral seal 50. In an alternative embodiment, the side edges 70 of the flap reinforcing elements 68 are co-extensive with the longitudinal sides of the napkin 20 and the distal end 56 of flaps 34, 36. In such an embodiment, the flap reinforcing elements 68 would have only one boundary 69 that is internal to the peripheral seal 50.

Although FIG. 2a depicts a specific embodiment in which each flap reinforcing element 68 is fastened to the liquid-impervious barrier layer 48, it should be expressly understood that flap reinforcing elements that are secured to the sanitary napkin 20 in other locations remain within the scope of the invention. FIG. 2b, for example, depicts an embodiment in which the flap reinforcing elements 68 are fastened to the fluid-permeable cover layer 40. Alternatively, the flap reinforcing elements 68 may be disposed within the sanitary napkin 20 and therefore hidden to view. FIG. 2c, for example, shows flap reinforcing elements 68 that are disposed between the absorbent system 42 and the fluid-permeable cover layer 40. Similarly, FIG. 2d shows flap reinforcing elements 68 that are disposed between the absorbent system 42 and the liquid-impervious layer 48.

It could also be advantageous, in an embodiment where the flap reinforcing element 68 is disposed between the fluid-permeable cover layer 40 and the absorbent system 42, for the flap reinforcing element 68 to possesses fluid management attributes. For example, the flap reinforcing element 68 may be more dense than the fluid-permeable cover layer 40 and have a larger proportion of smaller pores than does the latter. In addition to reinforcing the sanitary napkin, the flap reinforcing element 68 can therefore also act as a transfer layer. However, the flap reinforcing element 68 must still provide sufficient reinforcement to the flaps 34, 36.

In order to manufacture the sanitary napkins 20 shown in FIGS. 1 and 2a-2d, the flap reinforcing elements 68 can be pre-fastened to either the liquid-impervious barrier layer 48 or the fluid permeable cover layer 40 in positions that will cause the flap reinforcing elements 68 to span across the areas of juncture 52 when the sanitary napkin 20 is complete. As such, during production of the sanitary napkins 20, the layers of the sanitary napkins can be handled in the usual manner, without having to alter the equipment for incorporating the flap reinforcing elements 68 into the production process.

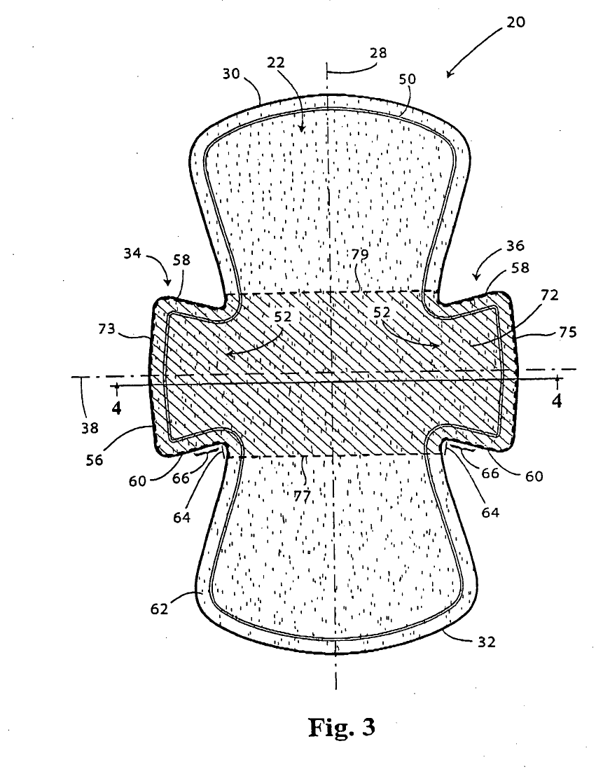

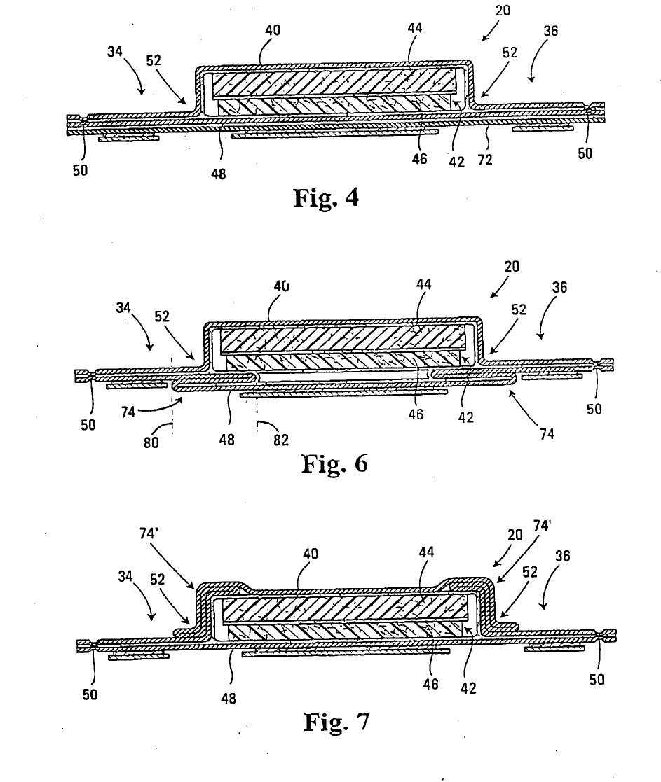

FIGS. 1 and 2a to 2d all depict embodiments in which the flap reinforcing elements extend in a direction that is substantially parallel with the imaginary longitudinal centerline 28. Other realizations wherein the flap reinforcing element extends in a direction that is essentially parallel to the imaginary transverse centerline 38, are equally possible. FIGS. 3 and 4 depict such an embodiment. As shown in FIG. 3, the sanitary napkin 20 includes a single flap reinforcing element 72 (shown in cross-hatching) which extends between the distal ends 56 of both flaps 34, 36. As such, flap reinforcing element 72 spans across the areas of juncture 52 associated with the flaps 34, 36 thereby bridging the main body 22 and the flaps 34, 36. FIG. 3 further shows that flap reinforcing element 72 is shaped such that it features edge portions 73, 75 that are respectively co-extensive with the distal ends 56 of flaps 34, 36 and co-extensive with the first and second segments 64, 66 of the continuous contour 62. In addition, flap reinforcing element 72 includes boundaries 77 and 79 that include portions that are internal to the peripheral seal 50.

Although not shown in FIGS. 3 and 4, the flap reinforcing element 72 can be positioned between the fluid permeable layer 40 and the fluid impervious layer 48. It could be advantageous, in the embodiment where the flap reinforcing element 72 is disposed transversely between the fluid-permeable cover layer 40 and the liquid-impervious barrier layer 48, for the flap reinforcing element 72 to possesses fluid management attributes, since the flap reinforcing element 72 covers the central area of the main body 22, which is typically the fluid impact zone.

In alternative embodiments where the transversely extending flap reinforcing element 72 is disposed between the liquid-impervious barrier layer 48 and the absorbent system 42 and wherein the former is made of breathable material (i.e., permeable to gases/vapors but impermeable to liquids), it could be advantageous for the flap reinforcing element 72 to comprise a material that is impermeable to both liquids and gases/vapors. Thus, enhanced protection against leaking is offered in an area that is coincident with bodily exudate discharge.

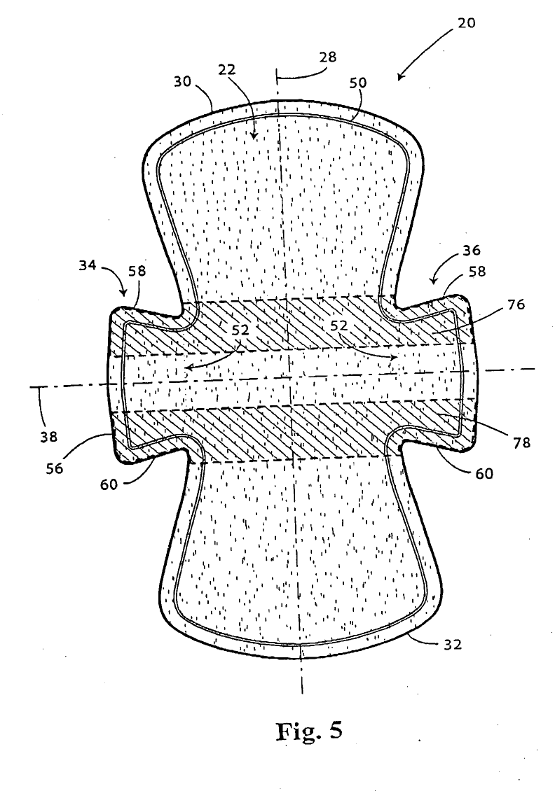

Although FIG. 3 depicts a single flap reinforcing element 72 extending in a direction that is essentially parallel to the imaginary transverse centerline 38, it should be specifically understood that any number of flap reinforcing elements extending in this same direction nonetheless remain within the scope of the present invention. FIG. 5, for example, shows a sanitary napkin having two flap reinforcing elements 76, 78. Each of the flap reinforcing elements 76, 78, however, respectively extends between the distal ends of both flaps 34, 36, thereby spanning across both areas of juncture 52. Although FIGS. 3 to 5 show transversely extending flap reinforcing elements 72, 76, 78 that are fastened on the liquid-impervious barrier layer 48, it should be understood that transversely extending flap reinforcing elements that are secured to the sanitary napkin 20 in other locations remains within the scope of the present invention.

The flap reinforcing elements, such as flap reinforcing elements 68, 72, 76 and 78 described above, may comprise a polymeric material, such as a polyolefin or polyester, or alternatively, may comprise a fibrous or cellulosic material. Preferably, the flap reinforcing elements 68, 72, 76 and 78 have a thickness in the range of 7×10⁻⁴ inches to 1.25×10⁻³ inches and have tensile strengths that are sufficient to cause the areas of juncture 52 to resist tearing when subjected to tensile forces typically applied by a user when attempting to remove the napkin from an undergarment without having previously detached the flaps.

Each of the above embodiments describe flap reinforcing elements that include a single layer. It should be expressly understood, however, that multi-layered flap reinforcing elements are also encompassed by the present invention. For example, multiple plies of material may be combined together to form a multi-layer structure, or alternatively, a single piece of material can be folded in order to form a multi-layer structure.

In the examples of implementation depicted in FIGS. 6 and 7, the flap reinforcing elements 74 and 74′ are formed by folding an existing layer of the sanitary napkin 20 into multiple layers, in order to provide an area of increased strength. In the example of implementation shown in FIG. 6, the flap reinforcing layers 74 are each formed by folding the liquid-impervious barrier layer 48 along two longitudinally oriented fold lines such that the flap reinforcing elements 74 are three layers thick. The layers can be bound together via the use of adhesive, ultrasonic bonding, and the like. The flap reinforcing elements 74 are positioned such that they span across respective areas of juncture 52, thereby bridging the main body 22 and a respective flap. In addition, the flap reinforcing elements 74 include a first boundary 80 and a second boundary 82. Since the flap reinforcing element 74 is the portion of the liquid-impervious barrier layer that is three layers thick, the boundaries 80 and 82 of the flap reinforcing element are located where the three layers begin and where the three layers end. A top plan view of the sanitary napkin 20 shown in FIG. 6 would look the same as the top plan view of the sanitary napkin 20 shown in FIG. 1, wherein the cross-hatched area represents the three layers of liquid impervious barrier layer. As such, boundary 80 would correspond to boundary 71 and boundary 82 would correspond to boundary 69. Both boundaries 80 and 82 include portions that are internal to the peripheral seal 50. It should also be understood that in the case where the flap reinforcing element is two layers thick or more than three layers thick, the boundaries of the flap reinforcing element are located where the multiple layers begin and where the multiple layers end.

Although the flap reinforcing element 74 is made from the liquid-impervious barrier layer 48, it should be expressly understood that flap reinforcing elements that are made by folding other layers of the sanitary napkin 20 are also possible. As shown in FIG. 7, for example, the flap reinforcing element 74′ is formed by folding the fluid-permeable cover layer 40. Although the flap reinforcing element 74 and 74′ shown in FIGS. 6 and 7 extend in a direction that is substantially parallel to the imaginary longitudinal centerline 28, it should be expressly understood that a flap reinforcing element formed of a fold that extends in a direction that is substantially parallel to the imaginary transverse centerline 38 also remains within the scope of the present invention.

Although various embodiments have been illustrated, this was for the purpose of describing, but not limiting, the invention. Various modifications will become apparent to those skilled in the art and are within the scope of this invention, which is defined more particularly by the attached claims.

1) A sanitary napkin adapted to be worn in the crotch portion of an undergarment, said sanitary napkin comprising:
a main body including:
a) a fluid-permeable layer facing toward the wearer when the sanitary napkin is placed in the undergarment;
b) a liquid-impervious layer;
c) an absorbent system for absorbing liquid, said absorbent system being located between said fluid-permeable layer and said liquid-impervious layer;
d) a pair of opposite longitudinal side edges;
e) a pair of opposite transverse side edges;
f) a peripheral seal for joining said fluid-permeable layer and said liquid-impervious layer together
at least one flap attached to said main body at an area of juncture, said at least one flap having two sides and a distal end remote from said area of juncture, said at least one flap and said main body together defining a continuous contour, said continuous contour comprising a first segment and a second segment, said first segment extending along one of said longitudinal side edges from said area of juncture toward one of said transverse side edges and said second segment extending along one of said sides of said at least one flap from said area of juncture toward said distal end; and
a flap reinforcing element that spans across said area of juncture thereby bridging said main body and said flap, said flap reinforcing element having:
a) an edge portion that is co-extensive with said first segment and said second segment of said continuous contour; and
b) a boundary that is internal to said peripheral seal.

2) A sanitary napkin as defined in claim 1, wherein said flap reinforcing element extends along said longitudinal centerline.

3) A sanitary napkin as defined in claim 1, wherein said flap reinforcing element extends along said transverse centerline.

4) A sanitary napkin as defined in claim 1, wherein said flap reinforcing element is fastened to said fluid-permeable layer.

5) A sanitary napkin as defined in claim 1, wherein said flap reinforcing element is fastened to said liquid-impervious layer.

6) A sanitary napkin as defined in claim 1, wherein said flap reinforcing element is disposed between said fluid-permeable layer and said liquid-impervious layer.

7) A sanitary napkin as defined in claim 1, wherein said absorbent system is a multi-layer structure including a first layer and a second layer, said flap reinforcing element being disposed between said first layer and said second layer.

8) A sanitary napkin as defined in claim 1, wherein said flap reinforcing element has an edge portion co-extensive with at least a portion of the distal end of said at least one flap.

9) A sanitary napkin as defined in claim 6, wherein said flap reinforcing element is more dense than said fluid-permeable layer.

10) A sanitary napkin as defined in claim 9, wherein said flap reinforcing element is characterized by a breathability that is less than that of said liquid-impervious layer.

11) A sanitary napkin as defined in claim 1, wherein said flap reinforcing element includes a fold.

12) A sanitary napkin as defined in claim 11, wherein said fold is formed on said liquid-impervious layer.

13) A sanitary napkin as defined in claim 11, wherein said fold is formed on said fluid-permeable layer.

14) A sanitary napkin as defined in claim 11, wherein said fold extends along said line of juncture.
